(19) United States
(12) Patent Application Publication
(10) Pub. No.: US 2022/0280110 A1
(43) Pub. Date: Sep. 8, 2022

(54) Data Aggregation and Power Distribution in Time Domain-Based Optical Measurement Systems

(71) Applicant: HI LLC, Los Angeles, CA (US)

(72) Inventors: Ryan Field, Culver City, CA (US); Jacob Dahle, Arlington, MA (US); Alex Borisevich, Lancaster, CA (US); Wingyuen Poon, Los Angeles, CA (US); Milin J. Patel, Anaheim, CA (US)

(21) Appl. No.: 17/680,828

(22) Filed: Feb. 25, 2022

Related U.S. Application Data
(60) Provisional application No. 63/156,793, filed on Mar. 4, 2021.

(51) Int. Cl. A61B 5/00

(52) U.S. Cl. CPC A61B 5/6803; A61B 5/0075; A61B 2560/0443

(57) ABSTRACT

An illustrative optical measurement system may include a primary controller; a plurality of secondary controllers communicatively coupled to the primary controller; and a plurality of modules, each module included in the plurality of modules comprising: a light source configured to emit light directed at a target, and a plurality of detectors configured to detect photon arrival times for the light after the light is scattered by the target; wherein: the plurality of modules is divided into a plurality of module subsets, and each module subset included in the plurality of module subsets is communicatively coupled to a respective secondary controller included in the plurality of secondary controllers.

### RELATED APPLICATIONS

The present application claims priority under 35 U.S.C. § 119(e) to U.S. Provisional Patent Application No. 63/156,793, filed Mar. 4, 2021, and incorporated herein by reference in its entirety.

### BACKGROUND INFORMATION

Time domain-based optical measurement systems (e.g., near-infrared spectroscopy (TD-NIRS) systems) have been considered the gold standard for optical brain imaging systems given their increased information content over continuous wave (CW) systems. In time domain-based optical measurement systems, picosecond pulses of light are emitted into tissue, and arrival times of single photons are measured at nearby detectors. The distribution of photon arrival times can be parameterized to estimate tissue optical properties, such as absorption (μₐ) and reduced scattering (μₛ′) coefficients. The photon arrival times can also be used to localize changes in deeper tissues by analyzing the later-arriving photons (“gating”) or analyzing moments of the time of flight (ToF) distribution. Unfortunately, conventional time-domain optical measurement systems are expensive, complex, and have large form-factors, which prevent them from being portable, wearable, and widely adopted.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an exemplary optical measurement system.

FIG. 2 illustrates an exemplary detector architecture.

FIG. 3 illustrates an exemplary timing diagram for performing an optical measurement operation using an optical measurement system.

FIG. 4 illustrates a graph of an exemplary temporal point spread function that may be generated by an optical measurement system in response to a light pulse.

FIG. 5 shows an exemplary non-invasive wearable brain interface system.

FIG. 6A-6B show an exemplary optical measurement system.

FIG. 7 shows an illustrative modular assembly.

FIGS. 8A-8B show an exemplary implementation of the modular assembly of FIG. 7.

FIG. 9 shows an exemplary multimodal measurement system.

FIG. 10 shows an illustrative modular assembly.

FIG. 11 shows an illustrative hierarchical architecture.

FIG. 12 shows various components that may be included in a primary controller.

FIG. 13 shows an exemplary implementation of a hierarchical architecture in which a primary controller and a plurality of secondary controllers each includes a buffer.

FIG. 14 shows example measurement cycles.

FIG. 15 shows various example histogram data collection techniques.

### DETAILED DESCRIPTION

Time domain-based optical measurement systems described herein include modules that can be assembled to provide dense channel coverage over the entire human head. Each module may include one or more light sources (e.g., miniaturized laser drivers) and a plurality of specialized detectors. As such, the time domain-based optical measurement systems described herein are wearable and portable, thus enabling wide-spread adoption for various types of use (e.g., commercial use, research use, medical use, etc.).

In some examples, a hierarchical-based controller architecture may be used to facilitate optimized data aggregation and power distribution in a modular time domain-based optical measurement system. For example, an optical measurement system may include a primary controller, a plurality of secondary controllers communicatively coupled to the primary controller, and a plurality of modules. Each module may include one or more light sources configured to emit light directed at a target (e.g., the brain) and a plurality of detectors configured to detect photon arrival times for the light after the light is scattered by the target. The plurality of modules may be divided into a plurality of module subsets, where each module subset included in the plurality of module subsets is communicatively coupled to a respective secondary controller included in the plurality of secondary controllers. Using this architecture, measurement data (e.g., histogram data) may be funneled from each of the modules by way of the secondary controllers to the primary controller, which may aggregate the data and then transmit the data to a computing device (e.g., by way of a single universal serial type-C(USB-C) interface). Furthermore, the architecture allows for distribution of power to each of the modules.

As described herein, the hierarchical architecture may minimize the number of modules that need to share a single serial peripheral interface (SPI) bus, thereby reducing the bus loading and allowing for higher data transmission frequencies. This distribution of devices over many buses also makes it easier to operate consistently with modules removed from the system. These and other advantages and benefits of the present architectures, systems, and methods are described more fully herein.

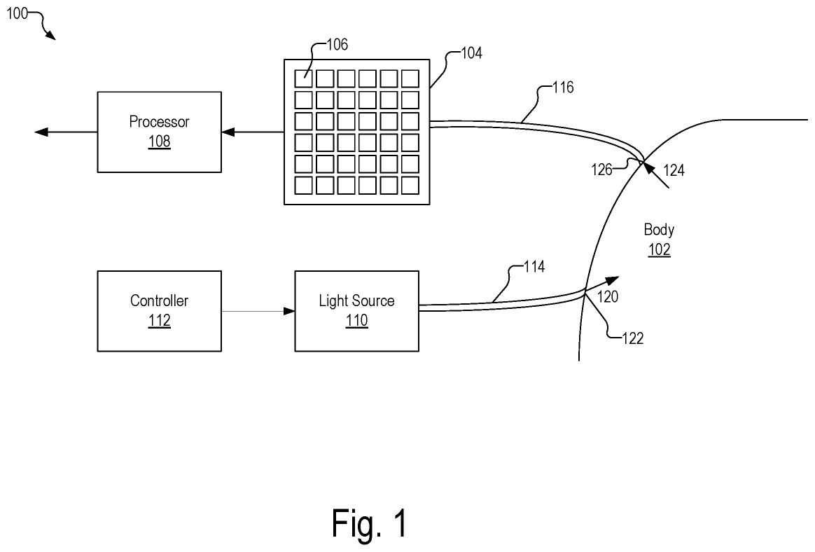

FIG. 1 shows an exemplary optical measurement system 100 configured to perform an optical measurement operation with respect to a body 102. Optical measurement system 100 may, in some examples, be portable and/or wearable by a user. Optical measurement systems that may be used in connection with the embodiments described herein are described more fully in U.S. patent application Ser. No. 17/176,315, filed Feb. 16, 2021 and published as US2021/0259638A1; U.S. patent application Ser. No. 17/176,309, filed Feb. 16, 2021 and published as US2021/0259614A1; U.S. patent application Ser. No. 17/176,460, filed Feb. 16, 2021 and issued as U.S. Pat. No. 11,096,620; U.S. patent application Ser. No. 17/176,470, filed Feb. 16, 2021 and published as US2021/0259619A1; U.S. patent application Ser. No. 17/176,487, filed Feb. 16, 2021 and published as US2021/0259632A1; U.S. patent application Ser. No. 17/176,539, filed Feb. 16, 2021 and published as US2021/0259620A1; U.S. patent application Ser. No. 17/176,560, filed Feb. 16, 2021 and published as US2021/0259597A1; U.S. patent application Ser. No. 17/176,466, filed Feb. 16, 2021 and published as US2021/0263320A1; Han Y. Ban, et al., “Kernel Flow: A High Channel Count Scalable TD-fNIRS System,” SPIE Photonics West Conference (Mar. 6, 2021); and Han Y. Ban, et al., “Kernel Flow: a high channel count scalable time-domain functional near-infrared spectroscopy system,” Journal of Biomedical Optics (Jan. 18, 2022), which applications and publications are incorporated herein by reference in their entirety.

In some examples, optical measurement operations performed by optical measurement system 100 are associated with a time domain-based optical measurement technique. Example time domain-based optical measurement techniques include, but are not limited to, time-correlated single-photon counting (TCSPC), time domain near infrared spectroscopy (TD-NIRS), time domain diffusive correlation spectroscopy (TD-DCS), and time domain Digital Optical Tomography (TD-DOT).

Optical measurement system 100 (e.g., an optical measurement system that is implemented by a wearable device or other configuration, and that employs a time domain-based (e.g., TD-NIRS) measurement technique) may detect blood oxygenation levels and/or blood volume levels by measuring the change in shape of laser pulses after they have passed through target tissue, e.g., brain, muscle, finger, etc. As used herein, a shape of laser pulses refers to a temporal shape, as represented for example by a histogram generated by a time-to-digital converter (TDC) coupled to an output of a photodetector, as will be described more fully below.

As shown, optical measurement system 100 includes a detector 104 that includes a plurality of individual photodetectors (e.g., photodetector 106), a processor 108 coupled to detector 104, a light source 110, a controller 112, and optical conduits 114 and 116 (e.g., light pipes). However, one or more of these components may not, in certain embodiments, be considered to be a part of optical measurement system 100. For example, in implementations where optical measurement system 100 is wearable by a user, processor 108 and/or controller 112 may in some embodiments be separate from optical measurement system 100 and not configured to be worn by the user.

Detector 104 may include any number of photodetectors 106 as may serve a particular implementation, such as 2ⁿ photodetectors (e.g., 256, 512, . . . , 16384, etc.), where n is an integer greater than or equal to one (e.g., 4, 5, 8, 10, 11, 14, etc.). Photodetectors 106 may be arranged in any suitable manner.

Photodetectors 106 may each be implemented by any suitable circuit configured to detect individual photons of light incident upon photodetectors 106. For example, each photodetector 106 may be implemented by a single photon avalanche diode (SPAD) circuit and/or other circuitry as may serve a particular implementation.

Processor 108 may be implemented by one or more physical processing (e.g., computing) devices. In some examples, processor 108 may execute instructions (e.g., software) configured to perform one or more of the operations described herein.

Light source 110 may be implemented by any suitable component configured to generate and emit light. For example, light source 110 may be implemented by one or more laser diodes, distributed feedback (DFB) lasers, super luminescent diodes (SLDs), light emitting diodes (LEDs), diode-pumped solid-state (DPSS) lasers, super luminescent light emitting diodes (sLEDs), vertical-cavity surface-emitting lasers (VCSELs), titanium sapphire lasers, micro light emitting diode (m LEDs), and/or any other suitable laser or light source configured to emit light in one or more discrete wavelengths or narrow wavelength bands. In some examples, the light emitted by light source 110 is high coherence light (e.g., light that has a coherence length of at least 5 centimeters) at a predetermined center wavelength. In some examples, the light emitted by light source 110 is emitted as a plurality of alternating light pulses of different wavelengths.

Light source 110 is controlled by controller 112, which may be implemented by any suitable computing device (e.g., processor 108), integrated circuit, and/or combination of hardware and/or software as may serve a particular implementation. In some examples, controller 112 is configured to control light source 110 by turning light source 110 on and off and/or setting an intensity of light generated by light source 110. Controller 112 may be manually operated by a user, or may be programmed to control light source 110 automatically.

Light emitted by light source 110 travels via an optical conduit 114 (e.g., a light pipe, a single-mode optical fiber, and/or or a multi-mode optical fiber) to body 102 of a subject. Body 102 may include any suitable turbid medium. For example, in some implementations, body 102 is a head or any other body part of a human or other animal. Alternatively, body 102 may be a non-living object. For illustrative purposes, it will be assumed in the examples provided herein that body 102 is a human head.

As indicated by an arrow 120, light emitted by light source 110 enters body 102 at a first location 122 on body 102. Accordingly, a distal end of optical conduit 114 may be positioned at (e.g., right above, in physical contact with, or physically attached to) first location 122 (e.g., to a scalp of the subject). In some examples, the light may emerge from optical conduit 114 and spread out to a certain spot size on body 102 to fall under a predetermined safety limit. At least a portion of the light indicated by arrow 120 may be scattered within body 102.

As used herein, “distal” means nearer, along the optical path of the light emitted by light source 110 or the light received by detector 104, to the target (e.g., within body 102) than to light source 110 or detector 104. Thus, the distal end of optical conduit 114 is nearer to body 102 than to light source 110, and the distal end of optical conduit 116 is nearer to body 102 than to detector 104. Additionally, as used herein, “proximal” means nearer, along the optical path of the light emitted by light source 110 or the light received by detector 104, to light source 110 or detector 104 than to body 102. Thus, the proximal end of optical conduit 114 is nearer to light source 110 than to body 102, and the proximal end of optical conduit 116 is nearer to detector 104 than to body 102.

As shown, the distal end of optical conduit 116 (e.g., a light pipe, a light guide, a waveguide, a single-mode optical fiber, and/or a multi-mode optical fiber) is positioned at (e.g., right above, in physical contact with, or physically attached to) output location 126 on body 102. In this manner, optical conduit 116 may collect at least a portion of the scattered light (indicated as light 124) as it exits body 102 at location 126 and carry light 124 to detector 104. Light 124 may pass through one or more lenses and/or other optical elements (not shown) that direct light 124 onto each of the photodetectors 106 included in detector 104.

Photodetectors 106 may be connected in parallel in detector 104. An output of each of photodetectors 106 may be accumulated to generate an accumulated output of detector 104. Processor 108 may receive the accumulated output and determine, based on the accumulated output, a temporal distribution of photons detected by photodetectors 106. Processor 108 may then generate, based on the temporal distribution, a histogram representing a light pulse response of a target (e.g., brain tissue, blood flow, etc.) in body 102. Example embodiments of accumulated outputs are described herein.

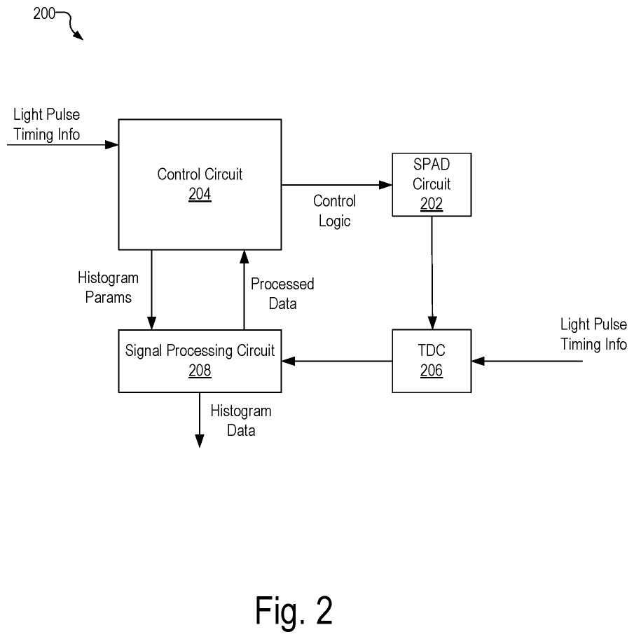

FIG. 2 illustrates an exemplary detector architecture 200 that may be used in accordance with the systems and methods described herein. As shown, architecture 200 includes a SPAD circuit 202 that implements photodetector 106, a control circuit 204, a time-to-digital converter (TDC) 206, and a signal processing circuit 208. Architecture 200 may include additional or alternative components as may serve a particular implementation.

In some examples, SPAD circuit 202 may include a SPAD and a fast gating circuit configured to operate together to detect a photon incident upon the SPAD. As described herein, SPAD circuit 202 may generate an output when SPAD circuit 202 detects a photon.

The fast gating circuit included in SPAD circuit 202 may be implemented in any suitable manner. For example, the fast gating circuit may include a capacitor that is pre-charged with a bias voltage before a command is provided to arm the SPAD. Gating the SPAD with a capacitor instead of with an active voltage source, such as is done in some conventional SPAD architectures, has a number of advantages and benefits. For example, a SPAD that is gated with a capacitor may be armed practically instantaneously compared to a SPAD that is gated with an active voltage source. This is because the capacitor is already charged with the bias voltage when a command is provided to arm the SPAD. This is described more fully in U.S. Pat. Nos. 10,158,038 and 10,424,683, which are incorporated herein by reference in their entireties.

In some alternative configurations, such as in configurations that implement the systems and methods described herein, SPAD circuit 202 does not include a fast gating circuit. In these configurations, the SPAD included in SPAD circuit 202 may be gated in any suitable manner or be configured to operate in a free running mode with passive quenching.

Control circuit 204 may be implemented by an application specific integrated circuit (ASIC) or any other suitable circuit configured to control an operation of various components within SPAD circuit 202. For example, control circuit 204 may output control logic that puts the SPAD included in SPAD circuit 202 in either an armed or a disarmed state.

In some examples, control circuit 204 may control a gate delay, which specifies a predetermined amount of time control circuit 204 is to wait after an occurrence of a light pulse (e.g., a laser pulse) to put the SPAD in the armed state. To this end, control circuit 204 may receive light pulse timing information, which indicates a time at which a light pulse occurs (e.g., a time at which the light pulse is applied to body 102). Control circuit 204 may also control a programmable gate width, which specifies how long the SPAD is kept in the armed state before being disarmed.

Control circuit 204 is further configured to control signal processing circuit 208. For example, control circuit 204 may provide histogram parameters (e.g., time bins, number of light pulses, type of histogram, etc.) to signal processing circuit 208. Signal processing circuit 208 may generate histogram data in accordance with the histogram parameters. In some examples, control circuit 204 is at least partially implemented by controller 112.

TDC 206 is configured to measure a time difference between an occurrence of an output pulse generated by SPAD circuit 202 and an occurrence of a light pulse. To this end, TDC 206 may also receive the same light pulse timing information that control circuit 204 receives. TDC 206 may be implemented by any suitable circuitry as may serve a particular implementation.

Signal processing circuit 208 is configured to perform one or more signal processing operations on data output by TDC 206. For example, signal processing circuit 208 may generate histogram data based on the data output by TDC 206 and in accordance with histogram parameters provided by control circuit 204. To illustrate, signal processing circuit 208 may generate, store, transmit, compress, analyze, decode, and/or otherwise process histograms based on the data output by TDC 206. In some examples, signal processing circuit 208 may provide processed data to control circuit 204, which may use the processed data in any suitable manner. In some examples, signal processing circuit 208 is at least partially implemented by processor 108.

In some examples, each photodetector 106 (e.g., SPAD circuit 202) may have a dedicated TDC 206 associated therewith. For example, for an array of N photodetectors 106, there may be a corresponding array of N TDCs 206. Alternatively, a single TDC 206 may be associated with multiple photodetectors 106. Likewise, a single control circuit 204 and a single signal processing circuit 208 may be provided for a one or more photodetectors 106 and/or TDCs 206.

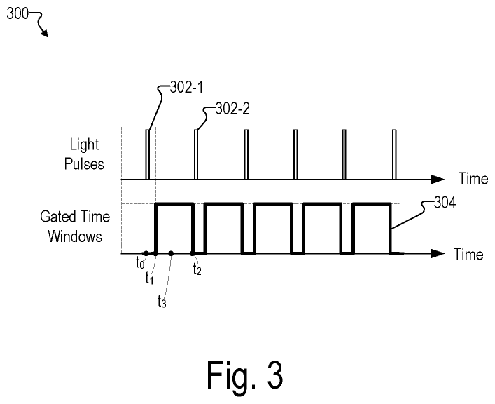

FIG. 3 illustrates an exemplary timing diagram 300 for performing an optical measurement operation using optical measurement system 100. The optical measurement operation may be performed in accordance with a time domain-based technique, such as TD-NIRS. Optical measurement system 100 may be configured to perform the optical measurement operation by directing light pulses (e.g., laser pulses) toward a target within a body (e.g., body 102). The light pulses may be short (e.g., 10-2000 picoseconds (ps)) and repeated at a high frequency (e.g., between 100,000 hertz (Hz) and 100 megahertz (MHz)). The light pulses may be scattered by the target and at least a portion of the scattered light may be detected by optical measurement system 100. Optical measurement system 100 may measure a time relative to the light pulse for each detected photon. By counting the number of photons detected at each time relative to each light pulse repeated over a plurality of light pulses, optical measurement system 100 may generate a histogram that represents a light pulse response of the target (e.g., a temporal point spread function (TPSF)). The terms histogram and TPSF are used interchangeably herein to refer to a light pulse response of a target.

Timing diagram 300 shows a sequence of light pulses 302 (e.g., light pulses 302-1 and 302-2) that may be applied to the target (e.g., tissue within a finger of a user, tissue within a brain of a user, blood flow, a fluorescent material used as a probe in a body of a user, etc.). Timing diagram 300 also shows a pulse wave 304 representing predetermined gated time windows (also referred as gated time periods) during which photodetectors 106 are gated ON to detect photons. As shown, light pulse 302-1 is applied at a time to. At a time t₁, a first instance of the predetermined gated time window begins. Photodetectors 106 may be armed at time t₁, enabling photodetectors 106 to detect photons scattered by the target during the predetermined gated time window. In this example, time t₁ is set to be at a certain time after time to, which may minimize photons detected directly from the laser pulse, before the laser pulse reaches the target. However, in some alternative examples, time t₁ is set to be equal to time to.

At a time t₂, the predetermined gated time window ends. In some examples, photodetectors 106 may be disarmed at time t₂. In other examples, photodetectors 106 may be reset (e.g., disarmed and re-armed) at time t₂ or at a time subsequent to time t₂. During the predetermined gated time window, photodetectors 106 may detect photons scattered by the target. Photodetectors 106 may be configured to remain armed during the predetermined gated time window such that photodetectors 106 maintain an output upon detecting a photon during the predetermined gated time window. For example, a photodetector 106 may detect a photon at a time t₃, which is during the predetermined gated time window between times t₁ and t₂. The photodetector 106 may be configured to provide an output indicating that the photodetector 106 has detected a photon. The photodetector 106 may be configured to continue providing the output until time t₂, when the photodetector may be disarmed and/or reset. Optical measurement system 100 may generate an accumulated output from the plurality of photodetectors. Optical measurement system 100 may sample the accumulated output to determine times at which photons are detected by photodetectors 106 to generate a TPSF.

As mentioned, in some alternative examples, photodetector 106 may be configured to operate in a free-running mode such that photodetector 106 is not actively armed and disarmed (e.g., at the end of each predetermined gated time window represented by pulse wave 304). In contrast, while operating in the free-running mode, photodetector 106 may be configured to reset within a configurable time period after an occurrence of a photon detection event (i.e., after photodetector 106 detects a photon) and immediately begin detecting new photons. However, only photons detected within a desired time window (e.g., during each gated time window represented by pulse wave 304) may be included in the TPSF.

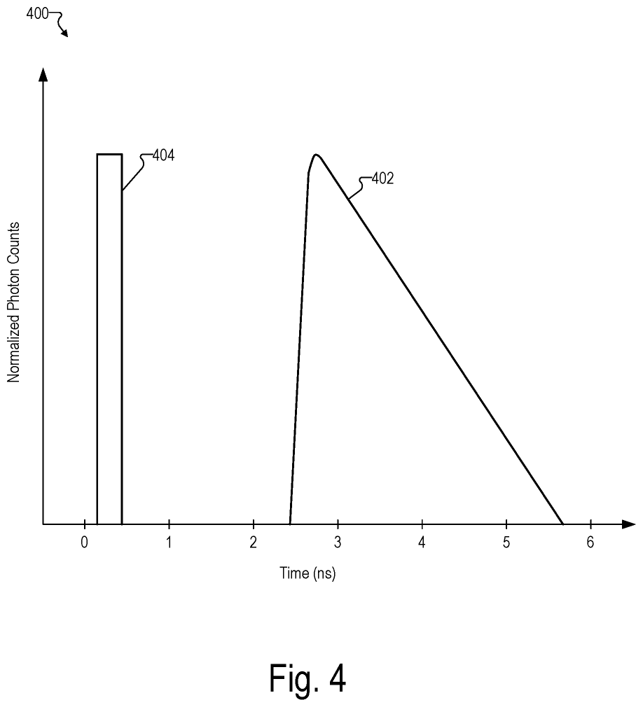

FIG. 4 illustrates a graph 400 of an exemplary TPSF 402 that may be generated by optical measurement system 100 in response to a light pulse 404 (which, in practice, represents a plurality of light pulses). Graph 400 shows a normalized count of photons on a y-axis and time bins on an x-axis. As shown, TPSF 402 is delayed with respect to a temporal occurrence of light pulse 404. In some examples, the number of photons detected in each time bin subsequent to each occurrence of light pulse 404 may be aggregated (e.g., integrated) to generate TPSF 402. TPSF 402 may be analyzed and/or processed in any suitable manner to determine or infer biological activity.

Optical measurement system 100 may be implemented by or included in any suitable device. For example, optical measurement system 100 may be included in a non-invasive wearable device (e.g., a headpiece) that a user may wear to perform one or more diagnostic, imaging, analytical, and/or consumer-related operations.

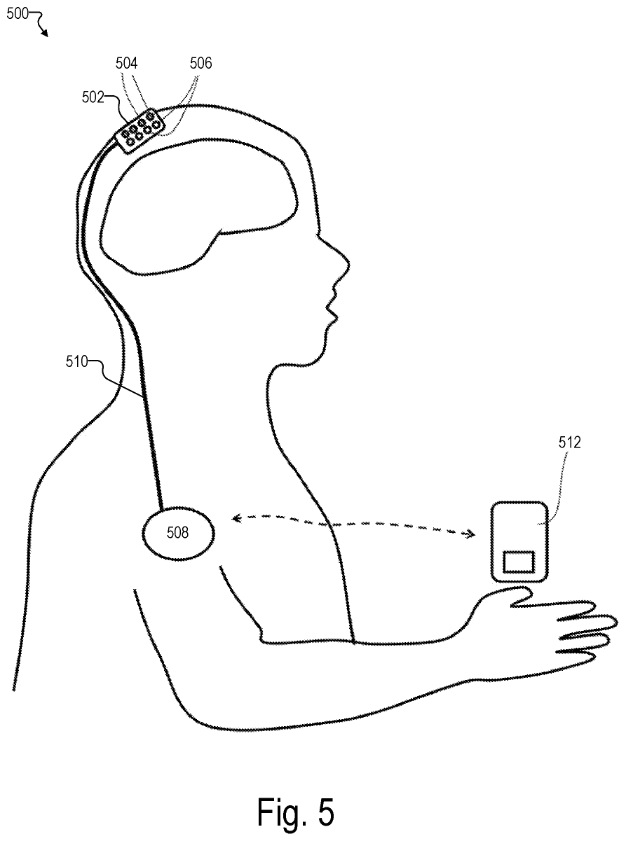

To illustrate, FIG. 5 shows an exemplary non-invasive wearable brain interface system 500 (“brain interface system 500”) that implements optical measurement system 100 (shown in FIG. 1). As shown, brain interface system 500 includes a head-mountable component 502 configured to be a wearable device (e.g., headgear) configured to be worn on a user's head. Head-mountable component 502 may be implemented by a cap shape that is worn on a head of a user. Alternative implementations of head-mountable component 502 include helmets, beanies, headbands, other hat shapes, or other forms conformable to be worn on a user's head, etc. For example, other forms conformable to be worm on the user's head include modular assemblies as will described more fully herein. Head-mountable component 502 may be made out of any suitable cloth, soft polymer, plastic, hard shell, and/or any other suitable material as may serve a particular implementation. Examples of headgears used with wearable brain interface systems are described more fully in U.S. Pat. No. 10,340,408, incorporated herein by reference in its entirety.

Head-mountable component 502 includes a plurality of detectors 504, which may implement or be similar to detector 104, and a plurality of light sources 506, which may be implemented by or be similar to light source 110. It will be recognized that in some alternative embodiments, head-mountable component 502 may include a single detector 504 and/or a single light source 506.

Brain interface system 500 may be used for controlling an optical path to the brain and for transforming photodetector measurements into an intensity value that represents an optical property of a target within the brain. Brain interface system 500 allows optical detection of deep anatomical locations beyond skin and bone (e.g., skull) by extracting data from photons originating from light source 506 and emitted to a target location within the user's brain, in contrast to conventional imaging systems and methods (e.g., optical coherence tomography (OCT)), which only image superficial tissue structures or through optically transparent structures.

Brain interface system 500 may further include a processor 508 configured to communicate with (e.g., control and/or receive signals from) detectors 504 and light sources 506 by way of a communication link 510. Communication link 510 may include any suitable wired and/or wireless communication link. Processor 508 may include any suitable housing and may be located on the user's scalp, neck, shoulders, chest, or arm, as may be desirable. In some variations, processor 508 may be integrated in the same assembly housing as detectors 504 and light sources 506.

As shown, brain interface system 500 may optionally include a remote processor 512 in communication with processor 508. For example, remote processor 512 may store measured data from detectors 504 and/or processor 508 from previous detection sessions and/or from multiple brain interface systems (not shown). Power for detectors 504, light sources 506, and/or processor 508 may be provided via a wearable battery (not shown). In some examples, processor 508 and the battery may be enclosed in a single housing, and wires carrying power signals from processor 508 and the battery may extend to detectors 504 and light sources 506. Alternatively, power may be provided wirelessly (e.g., by induction).

In some alternative embodiments, head mountable component 502 does not include individual light sources. Instead, a light source configured to generate the light that is detected by detectors 504 may be included elsewhere in brain interface system 500. For example, a light source may be included in processor 508 and coupled to head mountable component 502 through optical connections.

Optical measurement system 100 may alternatively be included in a non-wearable device (e.g., a medical device and/or consumer device that is placed near the head or other body part of a user to perform one or more diagnostic, imaging, and/or consumer-related operations). Optical measurement system 100 may alternatively be included in a sub-assembly enclosure of a wearable invasive device (e.g., an implantable medical device for brain recording and imaging).

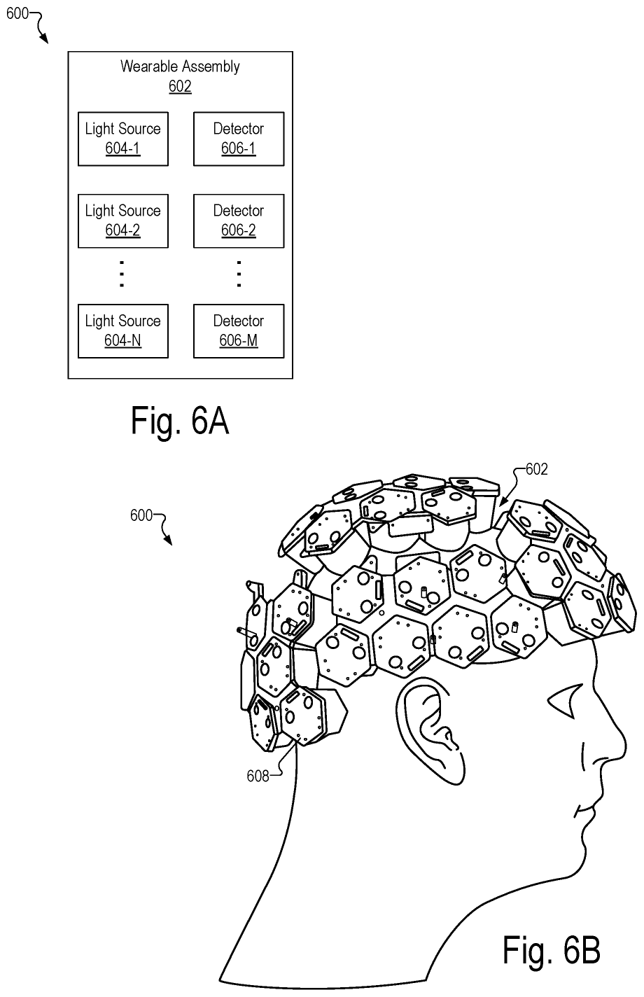

FIGS. 6A-6B shows an exemplary optical measurement system 600 in accordance with the principles described herein. Optical measurement system 600 may be an implementation of optical measurement system 100 and, as shown in FIG. 6A, includes a wearable assembly 602, which includes N light sources 604 (e.g., light sources 604-1 through 604-N) and M detectors 606 (e.g., detectors 606-1 through 606-M). Optical measurement system 600 may include any of the other components of optical measurement system 100 as may serve a particular implementation. N and M may each be any suitable value (i.e., there may be any number of light sources 604 and detectors 606 included in optical measurement system 600 as may serve a particular implementation).

Light sources 604 are each configured to emit light (e.g., a sequence of light pulses) and may be implemented by any of the light sources described herein. Detectors 606 may each be configured to detect arrival times for photons of the light emitted by one or more light sources 604 after the light is scattered by the target. For example, a detector 606 may include a photodetector configured to generate a photodetector output pulse in response to detecting a photon of the light and a TDC configured to record a timestamp symbol in response to an occurrence of the photodetector output pulse, the timestamp symbol representative of an arrival time for the photon (i.e., when the photon is detected by the photodetector).

Wearable assembly 602 may be implemented by any of the wearable devices, modular assemblies, and/or wearable units described herein. For example, as shown in FIG. 6B, wearable assembly 602 may be implemented by a wearable device (e.g., headgear) configured to be worn on a user's head. The TD-NIRS optical measurement system 600 shown in FIG. 6B may include a plurality of modules 608 arranged in a helmet design. Modules 608 may be organized on each side of the head, covering the frontal, parietal, temporal, and occipital cortices. Wearable assembly 602 may additionally or alternatively be configured to be worn on any other part of a user's body.

Optical measurement system 600 may be modular in that one or more components of optical measurement system 600 may be removed, changed out, or otherwise modified as may serve a particular implementation. As such, optical measurement system 600 may be configured to conform to three-dimensional surface geometries, such as a user's head, e.g., see FIG. 6B. Exemplary modular optical measurement systems comprising a plurality of wearable modules are described in more detail in U.S. patent application Ser. No. 17/176,460, filed Feb. 16, 2021 and issued as U.S. Pat. No. 11,096,620, U.S. patent application Ser. No. 17/176,470, filed Feb. 16, 2021 and published as US2021/0259619A1, U.S. patent application Ser. No. 17/176,487, filed Feb. 16, 2021 and published as US2021/0259632A1, U.S. patent application Ser. No. 17/176,539, filed Feb. 16, 2021 and published as US2021/0259620A1, U.S. patent application Ser. No. 17/176,560, filed Feb. 16, 2021 and published as US2021/0259597A1, and U.S. patent application Ser. No. 17/176,466, filed Feb. 16, 2021 and published as US2021/0263320A1, which applications are incorporated herein by reference in their respective entireties.

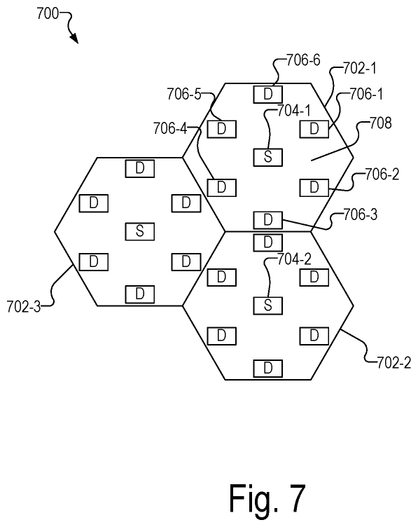

FIG. 7 shows an illustrative modular assembly 700 that may implement optical measurement system 600. Modular assembly 700 is illustrative of the many different implementations of optical measurement system 600 that may be realized in accordance with the principles described herein.

As shown, modular assembly 700 includes a plurality of modules 702 (e.g., modules 702-1 through 702-3). While three modules 702 are shown to be included in modular assembly 700, in alternative configurations, any number of modules 702 (e.g., a single module unit up to sixteen or more module units) may be included in modular assembly 700.

Each module unit 702 includes a light source (e.g., light source 704-1 of module 702-1 and light source 704-2 of module 702-2) and a plurality of detectors (e.g., detectors 706-1 through 706-6 of module 702-1). In the particular implementation shown in FIG. 7, each module unit 702 includes a single light source and six detectors. Each light source is labeled “S” and each detector is labeled “D”.

Each light source depicted in FIG. 7 may be implemented by one or more light sources similar to light source 110 and may be configured to emit light directed at a target (e.g., the brain).

Each light source depicted in FIG. 7 may be located at a center region of a surface of the light source's corresponding module. For example, light source 704-1 is located at a center region of a surface 708 of module 702-1. In alternative implementations, a light source of a module may be located away from a center region of the module.

Each detector depicted in FIG. 7 may implement or be similar to detector 104 and may include a plurality of photodetectors (e.g., SPADs) as well as other circuitry (e.g., TDCs), and may be configured to detect arrival times for photons of the light emitted by one or more light sources after the light is scattered by the target.

The detectors of a module may be distributed around the light source of the module. For example, detectors 706 of module 702-1 are distributed around light source 704-1 on surface 708 of module 702-1. In this configuration, detectors 706 may be configured to detect photon arrival times for photons included in light pulses emitted by light source 704-1. In some examples, one or more detectors 706 may be close enough to other light sources to detect photon arrival times for photons included in light pulses emitted by the other light sources. For example, because detector 706-3 is adjacent to module 702-2, detector 706-3 may be configured to detect photon arrival times for photons included in light pulses emitted by light source 704-2 (in addition to detecting photon arrival times for photons included in light pulses emitted by light source 704-1).

In some examples, the detectors of a module may all be equidistant from the light source of the same module. In other words, the spacing between a light source (i.e., a distal end portion of a light source optical conduit) and the detectors (i.e., distal end portions of optical conduits for each detector) are maintained at the same fixed distance on each module to ensure homogeneous coverage over specific areas and to facilitate processing of the detected signals. The fixed spacing also provides consistent spatial (lateral and depth) resolution across the target area of interest, e.g., brain tissue. Moreover, maintaining a known distance between the light source, e.g., light emitter, and the detector allows subsequent processing of the detected signals to infer spatial (e.g., depth localization, inverse modeling) information about the detected signals. Detectors of a module may be alternatively disposed on the module as may serve a particular implementation.

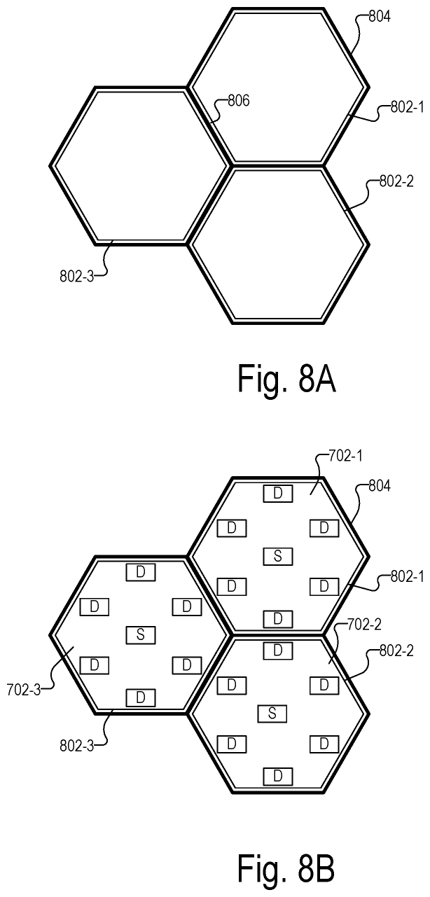

In FIG. 7, modules 702 are shown to be adjacent to and touching one another. Modules 702 may alternatively be spaced apart from one another. For example, FIGS. 8A-8B show an exemplary implementation of modular assembly 700 in which modules 702 are configured to be inserted into individual slots 802 (e.g., slots 802-1 through 802-3, also referred to as cutouts) of a wearable assembly 804. In particular, FIG. 8A shows the individual slots 802 of the wearable assembly 804 before modules 702 have been inserted into respective slots 802, and FIG. 8B shows wearable assembly 804 with individual modules 702 inserted into respective individual slots 802.

Wearable assembly 804 may implement wearable assembly 602 and may be configured as headgear and/or any other type of device configured to be worn by a user.

As shown in FIG. 8A, each slot 802 is surrounded by a wall (e.g., wall 806) such that when modules 702 are inserted into their respective individual slots 802, the walls physically separate modules 702 one from another. In alternative embodiments, a module (e.g., module 702-1) may be in at least partial physical contact with a neighboring module (e.g., module 702-2).

Each of the modules described herein may be inserted into appropriately shaped slots or cutouts of a wearable assembly, as described in connection with FIGS. 8A-8B. However, for ease of explanation, such wearable assemblies are not shown in the figures.

As shown in FIGS. 7 and 8B, modules 702 may have a hexagonal shape. Modules 702 may alternatively have any other suitable geometry (e.g., in the shape of a pentagon, octagon, square, rectangular, circular, triangular, free-form, etc.).

In some examples, any of the optical measurement systems described herein may be implemented by a wearable multimodal measurement system configured to perform both optical-based brain data acquisition operations and electrical-based brain data acquisition operations, such as any of the wearable multimodal measurement systems described in U.S. Patent Application Publication Nos. 2021/0259638 and 2021/0259614, which publications are incorporated herein by reference in their respective entireties.

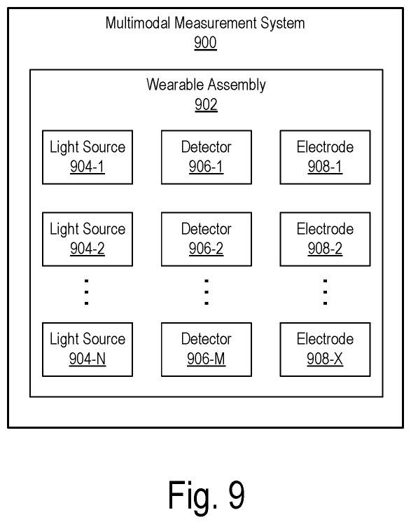

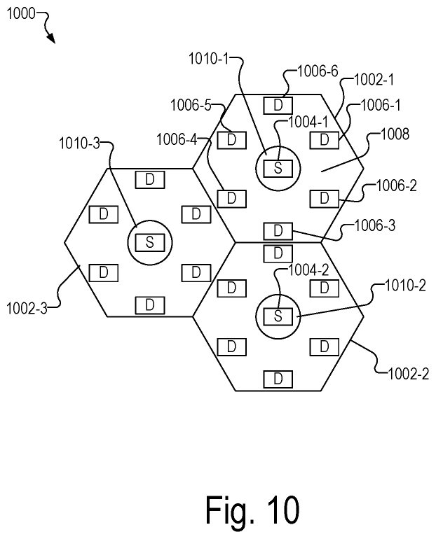

To illustrate, FIGS. 9-10 show various multimodal measurement systems that may implement optical measurement system 100. The multimodal measurement systems described herein are merely illustrative of the many different multimodal-based brain interface systems that may be used in accordance with the systems and methods described herein.

FIG. 9 shows an exemplary multimodal measurement system 900 in accordance with the principles described herein. Multimodal measurement system 900 may at least partially implement optical measurement system 100 and, as shown, includes a wearable assembly 902 (which may be similar to wearable assembly 602), which includes N light sources 904 (e.g., light sources 904-1 through 904-N, which are similar to light sources 604), M detectors 906 (e.g., detectors 906-1 through 906-M, which are similar to detectors 606), and X electrodes (e.g., electrodes 908-1 through 908-X). Multimodal measurement system 900 may include any of the other components of optical measurement system 100 as may serve a particular implementation. N, M, and X may each be any suitable value (i.e., there may be any number of light sources 904, any number of detectors 906, and any number of electrodes 908 included in multimodal measurement system 900 as may serve a particular implementation).

Electrodes 908 may be configured to detect electrical activity within a target (e.g., the brain). Such electrical activity may include electroencephalogram (EEG) activity and/or any other suitable type of electrical activity as may serve a particular implementation. In some examples, electrodes 908 are all conductively coupled to one another to create a single channel that may be used to detect electrical activity. Alternatively, at least one electrode included in electrodes 908 is conductively isolated from a remaining number of electrodes included in electrodes 908 to create at least two channels that may be used to detect electrical activity.

FIG. 10 shows an illustrative modular assembly 1000 that may implement multimodal measurement system 900. As shown, modular assembly 1000 includes a plurality of modules 1002 (e.g., modules 1002-1 through 1002-3). While three modules 1002 are shown to be included in modular assembly 1000, in alternative configurations, any number of modules 1002 (e.g., a single module up to sixteen or more modules) may be included in modular assembly 1000. Moreover, while each module 1002 has a hexagonal shape, modules 1002 may alternatively have any other suitable geometry (e.g., in the shape of a pentagon, octagon, square, rectangular, circular, triangular, free-form, etc.).

Each module 1002 includes a light source (e.g., light source 1004-1 of module 1002-1 and light source 1004-2 of module 1002-2) and a plurality of detectors (e.g., detectors 1006-1 through 1006-6 of module 1002-1). In the particular implementation shown in FIG. 10, each module 1002 includes a single light source and six detectors. Alternatively, each module 1002 may have any other number of light sources (e.g., two light sources) and any other number of detectors. The various components of modular assembly 1000 shown in FIG. 10 are similar to those described in connection with FIG. 7.

As shown, modular assembly 1000 further includes a plurality of electrodes 1010 (e.g., electrodes 1010-1 through 1010-3), which may implement electrodes 908. Electrodes 1010 may be located at any suitable location that allows electrodes 1010 to be in physical contact with a surface (e.g., the scalp and/or skin) of a body of a user. For example, in modular assembly 1000, each electrode 1010 is on a module surface configured to face a surface of a user's body when modular assembly 1000 is worn by the user. To illustrate, electrode 1010-1 is on surface 1008 of module 1002-1. Moreover, in modular assembly 1000, electrodes 1010 are located in a center region of each module 1002 and surround each module's light source 1004. Alternative locations and configurations for electrodes 1010 are possible.

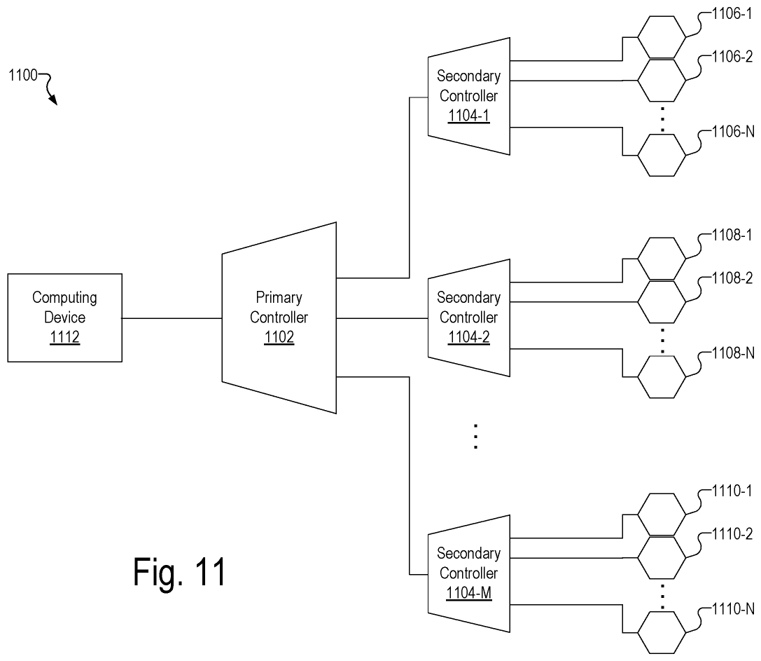

FIG. 11 shows an illustrative hierarchical architecture 1100 that may be used for any of the optical measurement systems described herein. As shown, architecture 1100 includes a primary controller 1102, a plurality of secondary controllers 1104 (e.g., secondary controllers 1104-1 through 1104-M) communicatively coupled to primary controller 1102, and a different module subset communicatively coupled to each secondary controller 1104. For example, a first module subset including modules 1106-1 through 1106-N is communicatively coupled to secondary controller 1104-1, a second module subset including modules 1108-1 through 1108-N is communicatively coupled to secondary controller 1104-2, and a third module subset including modules 1110-1 through 1110-N is communicatively coupled to secondary controller 1104-M.

Architecture 1100 may include any number of secondary controllers 1104 and corresponding module subsets. Likewise, each module subset may include any number of modules. For example, in one implementation, architecture 1100 may include four secondary controllers each communicatively coupled to a module subset that includes up to thirteen modules.

Secondary controllers 1104 may be communicatively coupled to the modules using any suitable interface. For example, each secondary controller 1104 may be communicatively coupled to the modules by way of a wired serial bus (e.g., a Serial Peripheral Interface (SPI) bus).

As shown, primary controller 1102 may also be communicatively coupled to a computing device 1112, e.g., by way of a universal serial type-C (USB-C) interface. In some examples, computing device 1112 is included in the optical measurement system (e.g., included in a wearable assembly that implements the optical measurement system). Alternatively, computing device 1112 may be separate from the optical measurement system (e.g., not included in a wearable assembly that implements the optical measurement system). Computing device 1112 may be implemented by a desktop computer, a mobile device (e.g., a laptop computer, a smartphone, or a tablet computer), a server, and/or any other type of computing device 1112 as may serve a particular implementation.

Each module shown in FIG. 11 may be implemented by any of the modules described herein. For example, each module may include a light source configured to emit light directed at a target (e.g. the brain) and a plurality of detectors configured to detect photon arrival times for the light after the light is scattered by the target. In some examples, the light source may include a first light source configured to emit light pulses at a first wavelength (e.g., red light at or around 6110 nm) and a second light source configured to emit light pulses at a second wavelength (e.g., infrared light at or around 850 nm). In some examples, the detectors are implemented by ASICs and/or other types of circuitry.

Primary controller 1102 may be implemented in any suitable manner. For example, primary controller 1102 may be implemented by a microcontroller unit (MCU) and/or any other processing device and/or circuitry configured to transmit commands, data, and power. Primary controller 1102 may include or more components configured to provide various types of functionality, as may serve a particular implementation.

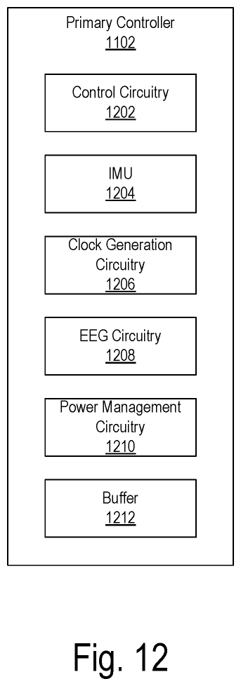

For example, FIG. 12 shows various components that may be included in primary controller 1102. The various components shown in FIG. 12 are merely illustrative of the many different components that may be included in primary controller 1102 as may serve a particular implementation.

As shown, primary controller 1102 may include control circuitry 1202, an inertial measurement unit (IMU) 1204, clock generation circuitry 1206, EEG circuitry 1208, power management circuitry 1210, and a buffer 1212.

Control circuitry 1202 may be configured to control an operation of secondary controllers 1104 and modules 1106-1110. For example, control circuitry 1202 may be configured to transmit commands to modules 1106-1110 directing one or more light sources to emit light and one or more detectors to detect photon arrival times.

IMU 1204 may be implemented in any suitable manner, such as by a 9-axis IMU, and may be configured to generate motion data that may be used for various purposes.

Clock generation circuitry 1206 may be configured to generate a global reference clock that may be distributed to each of the modules connected to secondary controllers 1104.

EEG circuitry 1208 may be implemented by a multi-channel (e.g., 8-channel) EEG amplifier and analog-to-digital converter (ADC) that may be used to connect to electrodes (e.g., active dry electrodes), such as any of the electrodes of the multimodal measurement systems described herein.

Power management circuitry 1210 may be configured to provide power to various components within the optical measurement system (e.g., secondary controllers 1104 and modules 1106-1110). For example, power management circuitry 1210 may handle the primary power negotiation for the Universal Serial Bus Power Delivery (USB-PD) standard and distribution of power to the rest of the optical measurement system.

Buffer 1212 may be implemented by any suitable storage device (e.g., one or more memory units) and may be used to temporarily store and/or aggregate measurement data (e.g., histogram data) generated by modules 1106-1110. Examples of this are described herein.

Returning to FIG. 11, secondary controllers 1104 may be implemented in any suitable manner. For example, secondary controllers 1104 may each be implemented by an MCU and/or any other processing device and/or circuitry configured to relay commands, data, and power. Secondary controllers 1104 may include or more components configured to provide various types of functionality, as may serve a particular implementation. For example, secondary controllers 1104 may each include a buffer, as described herein. Secondary controllers 1104 may, in some examples, further include an IMU and/or other circuitry described herein.

Primary controller 1102 and secondary controllers 1104 may serve as a funnel for collecting and combining data from each of the modules. Through this hierarchical architecture, full histogram frames may be streamed from each module and off of the system (e.g., to computing device 1112). Moreover, the hierarchical architecture may minimize the number of modules that need to share a single serial bus, thereby reducing the bus loading, and allowing for higher data transmission frequencies. This distribution of devices over many buses also makes it easier to operate consistently with modules removed from the system (e.g., the bus loading can be quite different for a single module versus four).

Various examples of primary controller 1102 controlling an operation of modules 1106-1110 will now be provided.

In some examples, primary controller 1102 may output, to a first module, a first command that directs the light source of the first module to emit a first series of light pulses at a first series of times. The first command may also direct the detectors of the first module to detect photon arrival times during time windows associated with the first series of times. Likewise, primary controller 1102 may output, to a second module, a second command that directs the light source of the second module to emit a second series of light pulses at a second series of times. The second command may also direct the detectors of the second module to detect photon arrival times during time windows associated with the second series of times. As described herein, data representative of the photon arrival times may be referred to herein as measurement data or histogram data.

In one implementation, the first module in the preceding example may be module 1106-1 and the second module may be module 1108-1. In this implementation, primary controller 1102 may transmit the first command to secondary controller 1104-1, which may relay the command to module 1106-1. Likewise, primary controller 1102 may transmit the second command to secondary controller 1104-2, which may relay the command to module 1108-1.

Alternatively, the first module in the preceding example may be module 1106-1 and the second module may be module 1106-2. In this implementation, primary controller 1102 may transmit the first and second commands to secondary controller 1104-1, which may relay the first and second commands to module 1106-1 and 1106-2, respectively.

In some examples, primary controller 1102 may coordinate measurement data collection so that modules (e.g., adjacent modules) do not interfere with each other. For example, in the preceding example, primary controller 1102 may ensure that the first series of times and the time windows associated with the first series of times are different from the second series of times and the time windows associated with the second series of times.

If modules are spaced far enough apart (e.g., more than a threshold distance), concurrent operation of the components on the modules will not interfere with one another. Hence, primary controller 1102 may allow such modules to operate concurrently. For example, in the preceding example, based on the first module being at least a threshold distance away from the second module, primary controller 1102 may allow for at least one time in the first series of times to be simultaneous to at least one time included in the second series of times.

In some examples, primary controller 1102 may facilitate cross-module measurements. For example, if the first module are relatively close to one another (e.g., adjacent to one another), primary controller 1102 may direct a detector included on the first module to detect photon arrival times associated with light output by a light source included on the second module. To illustrate, with reference to the preceding example, the first command may include a directing of the detector to detect photon arrival times during at least one time window included in the time windows associated with the second series of times. Furthermore, primary controller 1102 may prevent other detectors included on the first module from detecting photon arrival times associated with light output by the light source included on the second module. For example, with reference to the preceding example, the first command may include a directing of the other detectors to refrain from detecting photon arrival times during the at least one time window included in the time windows associated with the second series of times.

In some examples, the hierarchal architecture depicted in FIG. 11 may allow the optical measurement system to synchronously switch between operating modes (e.g., different detector or system configurations) quickly. As such, different instances of measurement data (e.g., different histograms) that are acquired may be collected with a different operating condition.

With this capability, the optical measurement system may be able to implement pseudo-random sampling patterns using unique configurations that are spatially spread across the head and/or implement non-uniform sampling of sensors on the head (e.g., focus measurements on a region of interest rather than uniformly sample the whole head). For example, with reference to the preceding example, based on a location of the first module, a location of the second module and a program setting of primary controller 1102, primary controller 1102 may specify that the first series of times has a greater number of times than the second series of times. In this manner, relatively more measurement data may be collected for a first region of the brain than a second region of the brain.

As another example, in cases where a module includes first and second light sources configured to output light at two different wavelengths (e.g., red light and infrared light) the optical measurement system may be able to accumulate different amounts of measurement data for each wavelength of (e.g., 60% of the time for the first wavelength and 40% of the time for the second wavelength).

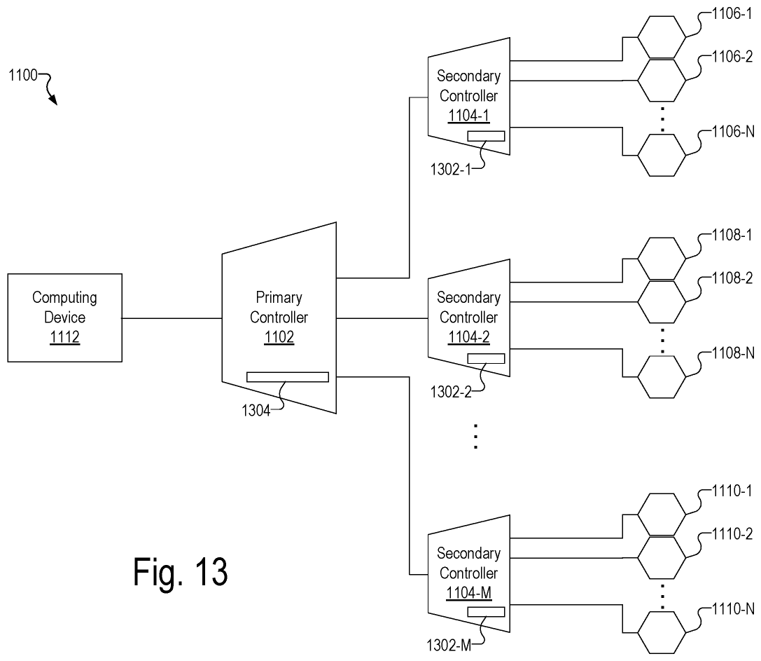

FIG. 13 shows an exemplary implementation of hierarchical architecture 1100 in which the secondary controllers 1104 and primary controller 1102 each includes a buffer. For example, secondary controllers 1104 may include buffers 1302-1 through 1302-M and primary controller 1102 may include buffer 1304. Buffers may, in some example, also be included in each of the modules.

Buffers 1302 and 1304 facilitate independent and synchronous accumulation and storage of measurement data by each of the modules for a single measurement cycle. As used herein, a measurement cycle refers to a time period during which histogram data representative of one or more histograms is collected.

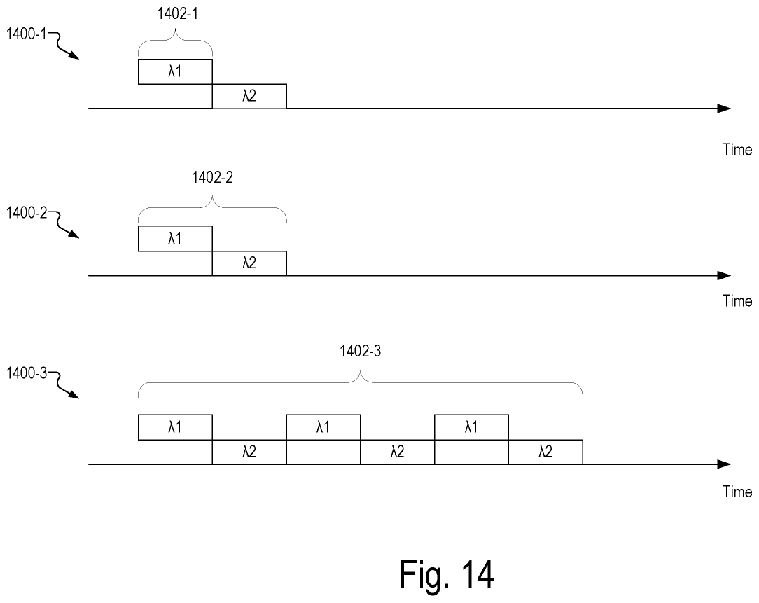

For example, FIG. 14 shows example measurement cycles that may be used in a configuration in which each module includes a first light source that emits light at a first wavelength and a second light source that emits light at a second wavelength. As shown, in a first configuration 1400-1, a measurement cycle 1402-1 may correspond to a single histogram cycle during which a single histogram associated with just one of the wavelengths is collected. In a second configuration 1400-2, a measurement cycle 1402-2 may correspond to two histogram cycles—a first histogram cycle during which a single histogram associated with the first wavelength is collected and a second histogram cycle during which a single histogram associated with the second wavelength is collected. In a third configuration 1400-3, a measurement cycle 1402-3 may correspond to a multi-histogram cycle during which a plurality of histograms associated with each of the first and second wavelengths are collected. A measurement cycle could alternatively be a period of sampling (e.g., multiple samples get buffered into a single packet) or it could be some multiple of histogram acquisitions packaged together (e.g., a histogram gathered from measurement during the first wavelength buffered with a histogram collected during measurement of the second wavelength. It will be recognized that this could be extended to configurations where there are more than two wavelengths for which histogram data is generated.

To illustrate the accumulation and storage of measurement data, the following example is provided. Module 1106-1 may be configured to generate, based on photon arrival times detected during time windows associated with a first series of light pulses generated by a first light source, first histogram data representative of one or more histograms. Module 1106-2 may likewise be configured to generate, based on photon arrival times detected during time windows associated with a second series of light pulses generated by a second light source, second histogram data representative of one or more histograms.

In this configuration, secondary controller 1104-1 may be configured to use buffer 1302-1 to aggregate the first histogram data and the second histogram data into aggregated histogram data. The aggregation may include buffering a measurement cycle of the first histogram data and the second histogram data into buffer 1302-1. Secondary controller 1104-1 may then relay the aggregated histogram data to primary controller 1102. Primary controller 1102 may buffer the aggregated histogram data in buffer 1304 and, in some examples, aggregate the aggregated histogram data with histogram data relayed thereto by the other secondary controllers 1104. Primary controller 1102 may be further configured to transmit the aggregated histogram data to computing device 1112.

In some examples, primary controller 1102 may direct the first and second modules (e.g., modules 1106-1 and 1106-2) to generate the first and second histogram data in an interleaved manner. In this manner, histograms associated with first and second wavelengths, for example, may be based on a series of smaller histograms that are collected in short intervals. This may allow for a more accurate measure of the average properties of the sample during the entire measurement period over which the two histograms are collected.

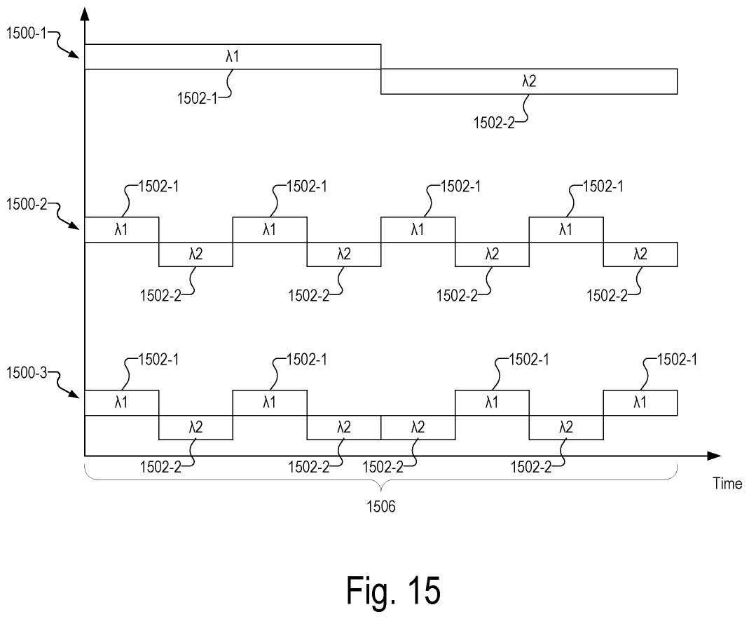

To illustrate, FIG. 15 shows a first configuration 1500-1 in which first histogram data 1502-1 corresponding to a first wavelength and second histogram data 1502-2 corresponding to a second wavelength are collected sequentially in a non-interleaved manner during a measurement cycle 1506. In contrast, configurations 1500-2 and 1500-3 illustrate interleaved collection of first and second histogram data 1502-1 and 1502-2 during the same measurement cycle 1506. As shown, in both configurations 1500-2 and 1500-3, the interleaved collection includes alternating between collecting different portions of first and second histogram data 1502-1 and 1502-2. The different portions may be aggregated upon completion of measurement cycle 1506.

By buffering the histogram data, the timing requirements for data acquisition may be decoupled from the timing requirements for reading out the data (histograms). Hence, the optical measurement system may be able to precisely time the start and end of each histogram acquisition, store and prepare the data for readout, and immediately start recording the next histogram, regardless of whether the last histogram has been read out. This allows for the timing between a secondary controller its corresponding modules to be relaxed and robust.

In some examples, a microcontroller and/or buffer may be included in each module as well. This may allow for added buffering, which can be used to gather and buffer data from each of the detectors contained within a module. This may also allow for complex data gathering schemes, for example, gathering all data for a pattern of cycles where lasers from some modules are fired while detectors within one or more other modules are measuring photons.

In some examples, measurement data may be encrypted at the module, at the secondary controller, and/or at the primary controller. All data that is transmitted across the physical layer (e.g., wires) throughout the system may therefore be encrypted. In cases where the measurement data is encrypted at the module, the modules may be designed with enough buffer space to store histograms from multiple cycles or full patterns, as no aggregation of encrypted data may be subsequently performed without first decrypting the data.

Hierarchical architecture 1100 may also simplify and improve performance of a power distribution network for the optical measurement system. Point of load power conversion may be balanced with thermal management to keep power regulators that dissipate energy away from the modules with thermally sensitive lasers and detectors. For example, a 20V rail may be distributed from the USB-PD input and regulated to an intermediate voltage at the secondary controller. This intermediate voltage may then be regulated down to the many final rails that are needed at the module. An added benefit of this architecture is that it minimizes the number of conductors that are needed to connect each module to the secondary controllers.

To illustrate, in some examples, primary controller 1102 may receive a voltage from a power source and distribute the voltage to secondary controllers 1104. Each secondary controller may be configured to regulate the voltage to an intermediate voltage and provide the intermediate voltage to each respective plurality of module subsets. Each module may then regulate the intermediate to a detector voltage.

An illustrative optical measurement system may include a primary controller; a plurality of secondary controllers communicatively coupled to the primary controller; and a plurality of modules, each module included in the plurality of modules comprising: a light source configured to emit light directed at a target, and a plurality of detectors configured to detect photon arrival times for the light after the light is scattered by the target; wherein: the plurality of modules is divided into a plurality of module subsets, and each module subset included in the plurality of module subsets is communicatively coupled to a respective secondary controller included in the plurality of secondary controllers.

An illustrative wearable device may include a wearable assembly; a plurality of modules each configured to be selectively inserted into the wearable assembly, each module included in the plurality of modules comprising: a light source configured to emit light directed at a target, and a plurality of detectors configured to detect photon arrival times for the light after the light is scattered by the target; a primary controller; and a plurality of secondary controllers communicatively coupled to the primary controller; wherein: the plurality of modules is divided into a plurality of module subsets, and each module subset included in the plurality of module subsets is communicatively coupled to a respective secondary controller included in the plurality of secondary controllers.

An illustrative method may include transmitting, by a primary controller by way of a first secondary controller, a first command to a first module that directs a light source of the first module to emit a first series of light pulses at a first series of times and directs a plurality of detectors of the first module to detect photon arrival times during time windows associated with the first series of times; transmitting, by the primary controller by way of a second secondary controller, a second command to a second module that directs a light source of the second module to emit a second series of light pulses at a second series of times and directs a plurality of detectors of the second module to detect photon arrival times during time windows associated with the second series of times; collecting, by the primary controller by way of the first secondary controller; first histogram data from the first module, the first histogram data based on the photon arrival times detected by the plurality of detectors of the first module; and collecting, by the primary controller by way of the second secondary controller; second histogram data from the second module, the second histogram data based on the photon arrival times detected by the plurality of detectors of the second module.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. An optical measurement system comprising:
a primary controller;
a plurality of secondary controllers communicatively coupled to the primary controller; and
a plurality of modules, each module included in the plurality of modules comprising:
a light source configured to emit light directed at a target, and
a plurality of detectors configured to detect photon arrival times for the light after the light is scattered by the target;
wherein:
the plurality of modules is divided into a plurality of module subsets, and
each module subset included in the plurality of module subsets is communicatively coupled to a respective secondary controller included in the plurality of secondary controllers.

2. The optical measurement system of claim 1, wherein:
the plurality of modules comprises a first module and a second module; and
the primary controller is configured to:
output, to the first module, a first command that directs the light source of the first module to emit a first series of light pulses at a first series of times and directs the plurality of detectors of the first module to detect photon arrival times during time windows associated with the first series of times; and
output, to the second module, a second command that directs the light source of the second module to emit a second series of light pulses at a second series of times and directs the plurality of detectors of the second module to detect photon arrival times during time windows associated with the second series of times.

3. The optical measurement system of claim 2, wherein the first series of times and the time windows associated with the first series of times are different from the second series of times and the time windows associated with the second series of times.

4. The optical measurement system of claim 3, wherein:
the plurality of detectors of the first module comprises a first detector; and
the first command comprises a directing of the first detector to further detect photon arrival times during at least one time window included in the time windows associated with the second series of times.

5. The optical measurement system of claim 4, wherein:
the plurality of detectors of the first module comprises a second detector; and
the first command comprises a directing of the second detector to refrain from detecting photon arrival times during the at least one time window included in the time windows associated with the second series of times.

6. The optical measurement system of claim 2, wherein, based on the first module being at least a threshold distance away from the second module, the first series of times comprises at least one time that is simultaneous to a time included in the second series of times.

7. The optical measurement system of claim 2, wherein, based on a location of the first module, a location of the second module and a program setting of the primary controller, the first series of times comprises a greater number of times than the second series of times.

8. The optical measurement system of claim 2, wherein:
the light source comprises a first light source configured to emit light pulses at a first wavelength and a second light source configured to emit light pulses at a second wavelength; and
the directing the light source to emit light at the first series of times comprises directing, based on a program setting of the primary controller, the first light source to emit light at the first wavelength a greater number of times of the first series of times than the second light source emits light at the second wavelength.

9. The optical measurement system of claim 2, wherein:
the plurality of secondary controllers comprises a particular secondary controller communicatively coupled to the first module and the second module;
the first module is configured to generate, based on the photon arrival times detected during the time windows associated with the first series of light pulses, first histogram data;
the second module is configured to generate, based on the photon arrival times detected during the time windows associated with the second series of light pulses, second histogram data; and
the particular secondary controller is configured to:
aggregate the first histogram data and the second histogram data into aggregated histogram data; and
relay the aggregated histogram data to the primary controller.

10. The optical measurement system of claim 9, wherein the aggregating the first histogram data and the second histogram data into the aggregated histogram data comprises buffering the first histogram data and the second histogram data during a measurement cycle.

11. The optical measurement system of claim 10, wherein:
the light source comprises a first light source configured to emit light pulses at a first wavelength and a second light source configured to emit light pulses at a second wavelength; and
the measurement cycle comprises a histogram cycle corresponding to the first wavelength and the second wavelength.

12. The optical measurement system of claim 9, wherein the primary controller is configured to direct the first and second modules to generate the first and second histogram data in an interleaved manner.

13. The optical measurement system of claim 9, wherein the primary controller is configured to transmit the aggregated histogram data to a computing device.

14. The optical measurement system of claim 2, wherein:
the plurality of secondary controllers comprises a first secondary controller and a second secondary controller;
the first module is communicatively coupled to the first secondary controller;
the second module is communicatively coupled to the second secondary controller;
the primary controller is configured to provide the first command to the first secondary controller and the second command to the second secondary controller;
the first secondary controller is configured to relay the first command to the first module; and
the second secondary controller is configured to relay the first command to the second module.

15. The optical measurement system of claim 1, wherein the primary controller is configured to:
receive a voltage from a power source; and
distribute the voltage to the plurality of secondary controllers.

16. The optical measurement system of claim 15, wherein each secondary controller is configured to:
regulate the voltage to an intermediate voltage; and
provide the intermediate voltage to each respective plurality of module subsets.

17. The optical measurement system of claim 16, wherein each module is configured to regulate the intermediate voltage to a detector voltage.

18. The optical measurement system of claim 1, wherein the primary controller, the plurality of secondary controllers, and the plurality of modules are included in a wearable assembly.

19. The optical measurement system of claim 18, wherein the wearable assembly is configured to be worn on a head of a user.

20. A wearable device comprising:
a wearable assembly;
a plurality of modules each configured to be selectively inserted into the wearable assembly, each module included in the plurality of modules comprising:
a light source configured to emit light directed at a target, and
a plurality of detectors configured to detect photon arrival times for the light after the light is scattered by the target;
a primary controller; and
a plurality of secondary controllers communicatively coupled to the primary controller;
wherein:
the plurality of modules is divided into a plurality of module subsets, and
each module subset included in the plurality of module subsets is communicatively coupled to a respective secondary controller included in the plurality of secondary controllers.

21. The wearable device of claim 20, wherein the wearable assembly is configured to be worn on a head of a user.

22. The wearable device of claim 21, wherein:
the plurality of modules comprises a first module and a second module; and
the primary controller is configured to:
output, to the first module, a first command that directs the light source of the first module to emit a first series of light pulses at a first series of times and directs the plurality of detectors of the first module to detect photon arrival times during time windows associated with the first series of times; and
output, to the second module, a second command that directs the light source of the second module to emit a second series of light pulses at a second series of times and directs the plurality of detectors of the second module to detect photon arrival times during time windows associated with the second series of times.

23. The wearable device of claim 22, wherein the first series of times and the time windows associated with the first series of times are different from the second series of times and the time windows associated with the second series of times.

24. The wearable device of claim 23, wherein:
the plurality of detectors of the first module comprises a first detector; and
the first command comprises a directing of the first detector to further detect photon arrival times during at least one time window included in the time windows associated with the second series of times.

25. The wearable device of claim 24, wherein:
the plurality of detectors of the first module comprises a second detector; and
the first command comprises a directing of the second detector to refrain from detecting photon arrival times during the at least one time window included in the time windows associated with the second series of times.

26. The wearable device of claim 22, wherein, based on the first module being at least a threshold distance away from the second module, the first series of times comprises at least one time that is simultaneous to a time included in the second series of times.

27. The wearable device of claim 22, wherein, based on a location of the first module, a location of the second module and a program setting of the primary controller, the first series of times comprises a greater number of times than the second series of times.

28. The wearable device of claim 22, wherein:
the light source comprises a first light source configured to emit light pulses at a first wavelength and a second light source configured to emit light pulses at a second wavelength; and
the directing the light source to emit light at the first series of times comprises directing, based on a program setting of the primary controller, the first light source to emit light at the first wavelength a greater number of times of the first series of times than the second light source emits light at the second wavelength.

29. The wearable device of claim 22, wherein:
the plurality of secondary controllers comprises a particular secondary controller communicatively coupled to the first module and the second module;
the first module is configured to generate, based on the photon arrival times detected during the time windows associated with the first series of light pulses, first histogram data;
the second module is configured to generate, based on the photon arrival times detected during the time windows associated with the second series of light pulses, second histogram data; and
the particular secondary controller is configured to:
aggregate the first histogram data and the second histogram data into aggregated histogram data; and
relay the aggregated histogram data to the primary controller.

30. The wearable device of claim 29, wherein the aggregating the first histogram data and the second histogram data into the aggregated histogram data comprises buffering the first histogram data and the second histogram data during a measurement cycle.

31. The wearable device of claim 30, wherein:
the light source comprises a first light source configured to emit light pulses at a first wavelength and a second light source configured to emit light pulses at a second wavelength; and
the measurement cycle comprises a histogram cycle corresponding to the first wavelength and the second wavelength.

32. The wearable device of claim 29, wherein the primary controller is configured to direct the first and second modules to generate the first and second histogram data in an interleaved manner.

33. The wearable device of claim 29, wherein the primary controller is configured to transmit the aggregated histogram data to a computing device.

34. The wearable device of claim 22, wherein:
the plurality of secondary controllers comprises a first secondary controller and a second secondary controller;
the first module is communicatively coupled to the first secondary controller;
the second module is communicatively coupled to the second secondary controller;
the primary controller is configured to provide the first command to the first secondary controller and the second command to the second secondary controller;
the first secondary controller is configured to relay the first command to the first module; and
the second secondary controller is configured to relay the first command to the second module.

35. The wearable device of claim 20, wherein the primary controller is configured to:
receive a voltage from a power source; and
distribute the voltage to the plurality of secondary controllers.

36. The wearable device of claim 35, wherein each secondary controller is configured to:
regulate the voltage to an intermediate voltage; and
provide the intermediate voltage to each respective plurality of module subsets.

37. The wearable device of claim 36, wherein each module is configured to regulate the intermediate voltage to a detector voltage.

38. A method comprising:
transmitting, by a primary controller by way of a first secondary controller, a first command to a first module that directs a light source of the first module to emit a first series of light pulses at a first series of times and directs a plurality of detectors of the first module to detect photon arrival times during time windows associated with the first series of times;
transmitting, by the primary controller by way of a second secondary controller, a second command to a second module that directs a light source of the second module to emit a second series of light pulses at a second series of times and directs a plurality of detectors of the second module to detect photon arrival times during time windows associated with the second series of times;
collecting, by the primary controller by way of the first secondary controller; first histogram data from the first module, the first histogram data based on the photon arrival times detected by the plurality of detectors of the first module; and
collecting, by the primary controller by way of the second secondary controller; second histogram data from the second module, the second histogram data based on the photon arrival times detected by the plurality of detectors of the second module.

39. The method of claim 38, further comprising:
aggregating, by the primary controller, the first and second histogram data into aggregated histogram data; and
transmitting, by the primary controller, the aggregated histogram data to a computing device.
